(19) United States
(12) Patent Application Publication
(10) Pub. No.: US 2011/0034478 A1
(43) Pub. Date: Feb. 10, 2011

(54) Oral Pharmaceutical Compositions in a Solid Dispersion Comprising Preferably Posaconazole and HPMCAs

(75) Inventors: Larry Yun Fang, Plainsboro, NJ (US); Jiansheng Wan, Warren, NJ (US); David Harris, New Providence, NJ (US)

Correspondence Address: MERCK;PATENT DEPARTMENT (K-6-1, 1990), 2000 GALLOPING HILL ROAD, KENILWORTH, NJ, 07033-0530, US

(73) Assignee: Schering-Plough Corporation

(21) Appl. No.: 12/937,881

(22) PCT Filed: Apr. 15, 2009
(86) PCT No.: PCT/US09/40653

§ 371 Date: Oct. 14, 2010

Related U.S. Application Data
(60) Provisional application No. 61/045,177, filed on Apr. 15, 2008.

(51) Int. Cl. A61K 31/496; A61P 31/10

(52) U.S. Cl. 514/254.07

(57) ABSTRACT

The present invention provides a solid molecularly dispersed composition comprising a poorly water soluble and weakly basic azole antifungal compound and a pH sensitive polymer, pharmaceutical compositions, comprising the solid molecularly dispersed composition of the invention and methods of treating and/or preventing a fungal infection in a patient in need thereof comprising orally administering a pharmaceutical composition comprising a composition of the invention to a patient in need thereof. Preferably the antifungal, compound is posaconazole, and the pH sensitive polymer is HPMCAS.

### FIELD OF THE INVENTION

The present invention relates to pharmaceutical compositions comprising a weakly basic and poorly-aqueous soluble azole molecularly dispersed in a pH sensitive polymer prepared by spray-drying. The invention also relates to methods for treating and/or preventing fungal infections by orally administering said pharmaceutical compositions.

### BACKGROUND OF THE INVENTION

Identification of any publication in this section or any section of this application is not an admission that such publication is prior art to the present invention.

Weakly basic and poorly-aqueous soluble (at intestinal pH) drugs comprising an azole functional group often show poor bioavailability or irregular absorption. Although these drugs are often soluble in a low pH environment, such as the stomach (pH 1-2), these drugs are largely insoluble at a higher pH environment, as found in the small intestine (pH 5-7). Consequently, such drugs generally precipitate out of solution as they pass from the low pH environment of the stomach into the higher pH environment of the small intestine. Examples of such weakly basic and poorly-aqueous soluble azole drugs are difenamizole, epirizole, fluconazole, itraconazole, ketoconazole, letrozole, posaconazole, ravuconazole, saperconazole, terconazole, and voriconazole.

Certain of these weakly basic and poorly-aqueous soluble azole drugs, including, but not limited to, itraconazole, posaconazole and terconazole have been developed for treatment and/or prevention of fungal infections, including invasive fungal infections. Thus far, the development of these drugs has been problematic as their solubility in aqueous solution is highly pH-dependent which results in difficulties in providing sufficient and easily controlled bioavailability.

U.S. Pat. Nos. 5,703,079 and 5,661,151 disclose posaconazole, a broad spectrum anti-fungal agent, the structure of which is illustrated below:

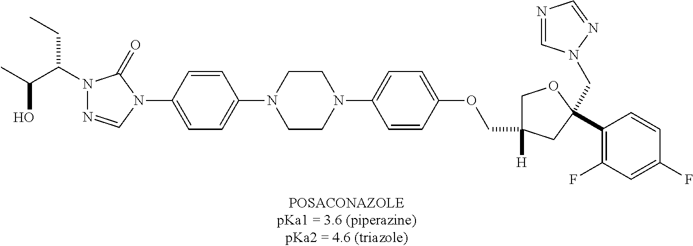

Posaconazole is partially solubilized in a strong acidic aqueous solution with a pH 1 or lower, where it has a solubility of about 790 μg/mL. In contrast, at pH>4, posaconazole has a solubility of less than 1 μg/mL in aqueous solution.

U.S. Pat. No. 5,834,472 ('472 Patent) describes a pharmaceutical composition of posaconazole, a non-ionic surfactant and a diluent. U.S. Pat. No. 5,972,381 ('381 Patent) describes pharmaceutical compositions of posaconazole and a soluble or insoluble polymer, such as, povidone or crospovidone, the components being present in particularly recited ratios. U.S. Pat. No. 5,846,971 ('971 Patent) describes pharmaceutical capsule compositions of posaconazole coated onto inert beads and a binder. None of the '472, '381, or the '971 Patents describe compositions which can provide an oral dosage form having low variability in bioavailability across a patient population.

U.S. Patent Application Publication No. US2003/0055067 describes pharmaceutical compositions of micronized particles of posaconazole together with a surfactant and thickening agent in the form of liquid suspensions, including an oral suspension commercially available under the tradename NOXAFIL™. Although the NOXAFIL™ suspension provides sufficient bioavailability, in order to insure adequate plasma concentrations in humans, it is recommended that NOXAFIL™ be administered in combination with food or a nutritional supplement and that it be administered several times daily. In addition, although a liquid oral suspension is advantageous for some patients, e.g., those who have difficulty swallowing pills, a solid dosage form is desired and/or needed for other situations. In particular, a solid dosage form is desirable for ease of portability, storage, and to promote patient compliance by avoiding the need for an additional dosing instrument, etc. In addition, there remains a need for a solid dosage form of posaconazole which provides sufficient bioavailability, with low variability across a patient population and which avoids the need for administration in combination with food. Likewise, a solid dosage form with sufficient bioavailability would provide a treatment regimen wherein the number of doses administered per day to achieve the desired therapeutic plasma concentration could be reduced.

Compositions having enhanced bioavailability which comprise pharmaceutical compounds and various polymers have been described in U.S. Pat. No. 7,235,260, issued Jun. 26, 2007 to Crew et al. (the '260 patent). In one example, the '260 patent describes glycogen phosphorlase inhibitors in hydroxypropylmethylcellulose and hydroxypropylmethylcellulose-derivative polymers. The compositions described in the '260 patent are prepared by spray-drying a solution containing a phosphorlase inhibitor and hydroxypropylmethylcellulose acetate succinate (HPMC-AS) dissolved in a common solvent. U.S. Pat. No. 6,881,745 (the '745 patent) issued Apr. 19, 2005 to Hayes et al., generally describes compositions comprising an azole antifungal compound and a polymer. The compositions described are prepared by dissolving the azole compound and polymer in a common solvent, for example, methylene chloride, chloroform, ethanol, methanol, isopropanol, ethylacetate, or acetone, or mixtures of two or more thereof, and forming a solid granular composition by spray-drying the solution using conventional spray-drying equipment. An example of an azole-containing composition described in the '745 patent is itraconazole with a hydroxypropylmethylcellulose-phthalate (HPMC-phthalate) polymer derivative prepared by spray-drying a solution containing the active pharmaceutical ingredient (API) and the polymer. These compositions are reported to show improvement in itraconazole bioavailability and elimination of a food effect connected with the administration of itraconazole.

There remains a need for pharmaceutical compositions with enhanced bioavailability that comprise a weakly basic and poorly-aqueous soluble azole, including posaconazole. Such compositions would reduce the dose and/or absorption variability required for the same or better therapeutic effect, reduce the cost of goods for the product, and/or reduce the dosing regimen. Solid dosage forms of such drugs would provide greater convenience for patients and hence promote patient compliance.

### SUMMARY OF THE INVENTION

These and other objectives and advantages are provided by the present invention which in one aspect is a composition suitable for oral administration to a patient that reduces variability in the bioavailability of posaconazole across a patient population and reduces the food effect observed with currently available oral dosage forms.

In some embodiments the composition is a molecular solid dispersion comprising a weakly basic and poorly-aqueous soluble azole in a stable amorphous form dissolved in a pH sensitive polymer. In some embodiments, the weakly basic and poorly-aqueous soluble azole is difenamizole, epirizole, fluconazole, itraconazole, ketoconazole, letrozole, ravuconazole, saperconazole, terconazole, or voriconazole, preferably the azole is fluconazole, itraconazole, ketoconazole, posaconazole, ravuconazole, saperconazole, terconazole, or voriconazole, more preferably the azole is itraconazole or terconazole. In some especially preferred embodiments the azole is posaconazole. In some embodiments the pH sensitive polymer is cellulose acetate phthalate, polyvinyl acetate phthalate, alginic acid, a methacrylic acid polymer, a carbomer, polacrillin, chitosan, or a hydroxypropylmethylcellulose-derivatve polymer, for example, hypromellose-phthalate or hypromellose acetate succinate (hydroxypropylmethylcellulose acetate succinate (HPMCAS)), prefererably the polymer is HPMCAS. In some especially preferred embodiments the composition to comprise posaconazole molecularly dispersed in HPMCAS.

In some embodiments where the composition comprises HPMCAS, the HPMCAS polymer selected is AQOAT® polymer grade L, grade M, or grade H (Shin-Etsu), or a combination of two or more thereof, preferably AQOAT grade L or grade M.

In some embodiments, the composition comprises posaconazole and HPMCAS in a ratio by weight of HPMCAS:posaconazole which is from about 1:1 to about 10:1. In some embodiments, the ratio by weight of HPMCAS:posaconazole is from about 2:1 to about 4:1, preferably about 3:1 ratio by weight of HPMCAS:posaconazole. In some embodiments it is preferred to provide a molecular solid dispersion having a weight percentage of HPMCAS polymer:posaconazole of from about 40 wt. % HPMCAS/60 wt. % posaconazole to about 95 wt. % HPMCAS/5 wt. % posaconazole.

In some embodiments it is preferred to select polymers that provide oral pharmaceutical compositions that limit the amount of posaconazole dissolved at pH<2 (e.g., as found in the stomach) and enhance the amount dissolved at pH≧5 (e.g., as found in the small intestines).

In some embodiments a composition of the invention comprising posaconazole placed maintained in an environment comprising an aqueous solution having a pH≧5 releases an amount of posaconazole that is at least 1.5 times greater, preferably at lease three times greater, than the amount of posaconazole released from the same composition when maintained in an environment comprising an aqueous solution having a pH<2.

The molecular solid dispersions provided herein (compositions of the invention) exhibit excellent mechanical and physical attributes necessary for preparing pharmaceutical dosage forms therefrom, for example, the compositions of the invention may be milled, blending, and incorporated into formulations suitable for the preparation of tablets, for example, preparation of tablets via direct compression. The compositions of the invention may thus be utilized as powders or granules directly or incorporated into a formulation comprising one or more pharmaceutically acceptable excipients, for example, in the preaparation of solid dosage forms, for example, capsules, tablets, granules, powders, and unit dose packets. It is believed that compositions of the invention can be employed to prepare dosage forms having a high drug loading, for example, when the composition comprise posaconaozle, a drug loading in excess of about 100 mg/unit dose. Pharmaceutical compositions comprising a molecular solid dispersion of the present invention are believed to provide a reduced food effect and to exhibit lower variability in bioavailability across a patient population when compared with the presently available commercial orally administered formulation.

In some embodiments, a molecular solid dispersion of the invention comprising posaconazole is stable for a period of at least one year when subjected to storage conditions of from about 15° C. to about 25° C. and from about 50% relative humidity to about 60% relative humidity, and is stable for a period of at least about 3 months when subjected to storage conditions about 40° C. and about 75% relative humidity.

In another aspect the invention provides a method for preparing a compositions of the invention (molecular solid dispersion of an azole antifungal compound). In some embodiments it is preferred to prepare an inventive composition by a process comprising: (a) dissolving an azole antifungal compound, in some especially preferred embodiments the azole antifungal compound is posaconazole, and a polymer, preferably HPMCAS in an organic solvent; and (b) evaporating the organic solvent. In certain embodiments, it is preferred to perform the dissolving step at a temperature of from about 25° C. to about 70° C. In certain embodiments it is preferred to carry out evaporating Step “b” by a spray drying technique. In certain embodiments wherein Step “b” is carried out by spray-drying, preferably spray-drying is performed at a temperature in the range of from about 30° C. to about 80° C. In certain embodiments it is preferred for the organic solvent to be methanol, ethanol, isopropanol, or acetone, or a combination of two or more thereof, preferably the organic solvent is methanol, ethanol or acetone.

Another aspect of the invention provides methods for the treatment of a patient and/or prevention of a condition due to fungal infection in a patient, for example, Otomycosis or Chromomycosis, or the treatment of an Asperillus or a Candida infection in a patient, or the prevention of an Asperillus or a Candida infection in a patient, the method comprising administering to a patient in need thereof a composition of the invention. Another aspect of the invention provides methods for the treatment and/or prevention of a fungal infection in a patient caused by zygomycetes, for example, Mucor, Rhizopus, or Rhizomucor etc., a fungal infection caused by a dermatophyte, for example, Tinea corporis, Tinea cruris, Tinea pedis, Tinea barbae, Tinea capitis, Tinea nigra, Tinea favosa, or Tinea Imbricata or any other agent associated with onychomycosis, the method comprising administering a composition of the invention to a patient in need thereof. In some embodiments it is preferred to administer a dose comprising a composition of the invention daily in a single dose, or a divided dose, for example, twice-a-day.

### BRIEF DESCRIPTION OF THE DRAWINGS

FIG. 1 shows dissolution profiles of two molecular solid dispersions of posaconazole with HPCMAS (grade L or grade M) according to the present invention designated herein as “Capsule A” and “Capsule B” respectively. As illustrated in FIG. 1, “% recovery” refers to the amount of posaconazole released from the samples relative to the total amount of posaconazole in the samples of solid molecular dispersions.

FIG. 2 shows a linear:linear graph of the mean plasma concentration of posaconazole over a time period of 120 hrs post-dose following a single oral administration of 60 mg posaconazole to male cynomolgus monkeys in either Capsule A, Capsule B, or, as a comparative example, an oral suspension of posaconazole.

FIG. 3 is a graph of the mean Cmax concentration of posaconazole following a single oral administration of 60 mg posaconazole to male cynomolgus monkeys in either Capsule A, Capsule B, or, as a comparative example, an oral suspension of posaconazole.

FIG. 4 is a graph of the mean exposure (AUC(tf)) of posaconazole following a single oral administration of 60 mg posaconazole to male cynomolgus monkeys in either Capsule A, Capsule B, or, as a comparative example, an oral suspension of posaconazole.

### DETAILED DESCRIPTION OF THE INVENTION

### Definitions

Unless defined otherwise, all technical and scientific terms used herein have the same meaning as those commonly understood by one of ordinary skill in the art to which this invention belongs. Although methods and materials similar or equivalent to those described herein can be used in the practice or testing of the present invention, suitable methods and materials are described below. The materials, methods and examples are illustrative only, and are not intended to be limiting. All publications, patents and other documents mentioned herein are incorporated by reference in their entirety.

As used herein, the term “AUC” is the area under the plasma concentration-time curve from time zero to a selected period of time studied. For example, AUC (4 h) means the area under the plasma concentration-time curve from time zero to 4 hours.

As used herein, the term “AUC(tf)” is the area under the plasma concentration-time curve from time zero to the time of the final quantifiable sample.

As used herein, the term “CL/F” is the apparent total clearance of the drug from plasma after oral administration. CL/F is calculated by dividing the dose administered by the AUC.

The term “patient” refers to an animal including a mammal (e.g., a human).

The term “pharmaceutically acceptable excipient” refers to those non-therapeutically active constituents of a dosage form which are generally recognized as safe for human ingestion.

The term “treating” or “treatment” is intended to mean therapeutic or prophylactic mitigation or alleviating one or more symptoms associated with a disease state.

The term “pharmacokinetics” refers to the process by which a drug is absorbed, distributed, metabolized, and eliminated by the body. Pharmacokinetic parameters include, but are not limited to, “maximum plasma concentration” or “Cmax,” “area under the plasma concentration time curve” or “AUC,” and “time to Cmax” or “Tmax.”

As used herein, the term “t1/2” refers to the half-life of the drug.

### Molecular Solid Dispersion Pharmaceutical Compositions

In some especially preferred embodiments the present invention provides oral pharmaceutical compositions comprising the solid molecular dispersion of the invention comprising posaconazole and HPMCAS that when maintained in an aqueous environment having a pH which is more acidic than about pH 2 (e.g., as found in the stomach), the pharmaceutical compositions release only a limited amount of posaconazole and when maintained in an aqueous environment having a pH which is less acidic than about pH 5 (e.g., as found in the small intestines) they release a large amount of the posaconazole comprising the composition. In especially preferred embodiments, the molecular solid dispersions provided herein, posaconazole in a stable amorphous form is dissolved in HPMCAS. The molecular solid dispersions provided herein exhibit excellent mechanical and physical attributes necessary for subsequent milling, blending, and tablet compression. Such molecular solid dispersions can be directly utilized as powders or granules. Alternatively, such molecular solid dispersions can be used to prepare solid dosage forms including capsules, tablets, granules, powders, and unit dose packets. In fact, the molecular solid dispersions provided herein are suitable for high drug loading dosage forms with ≧100 mg drug per unit dosage form. The pharmaceutical compositions of the present invention are believed to reduce the food effect in comparison with the food effect observed when orally administering the commercially available suspension and are believed to reduce the variability observed in bioavailablity across a patient population when compared with the commercially available oral dosage form.

In some embodiments, the present invention also provides oral pharmaceutical compositions comprising a pH sensitive polymer, preferably the polymer is cellulose acetate phthalate, polyvinyl acetate phthalate, alginic acid, a methacrylic acid polymer, a carbomer, polacrillin, chitosan, or a hydroxypropylmethylcellulose-derivatve polymer, for example, hypromellose-phthalate or hypromellose acetate succinate (hydroxypropylmethylcellulose acetate succinate (HPMCAS)), more prefererably the polymer is HPMCAS, and molecularly dispersed therein a weakly basic and poorly-aqueous soluble azole compound which is difenamizole, epirizole, fluconazole, itraconazole, ketoconazole, letrozole, ravuconazole, saperconazole, terconazole, or voriconazole, preferably fluconazole, itraconazole, ketoconazole, ravuconazole, saperconazole, terconazole, or voriconazole more preferably, the azole is itraconazole, posaconazole, or terconazole. In an especially preferred embodiment the azole is posaconazole and the polymer is HPMCAS.

Posaconazole has the following structure:

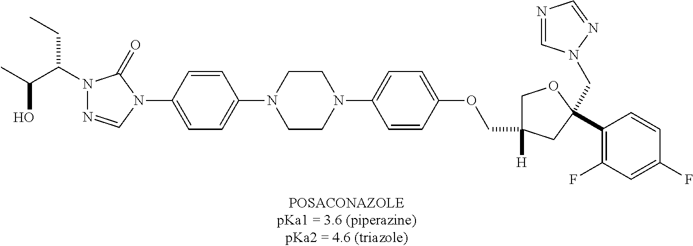

Posaconazole, available from Schering Corporation, Kenilworth, N.J., can be prepared according to Examples 24 and 32 of U.S. Pat. No. 5,661,151 and WO 95/17407. Posaconazole is partially solubilized in an aqueous solution having a pH which is pH 1 or more acid. In an aqueous solution of pH 1 posaconazole has a solubility of about 790 μg/mL. In contrast, in less acidic solutions, for example an aqueous solution of about pH 4 or less acidid, posaconazole has a solubility of less than 1 μg/mL.

Itraconazole, available from Janssen Pharmaceutica, N.V., Beerse, Belgium and described in U.S. Pat. No. 4,267,179, has a pKa=3.7 and is practically insoluble in water and diluted acidic solutions. Itraconazole has the following structure:

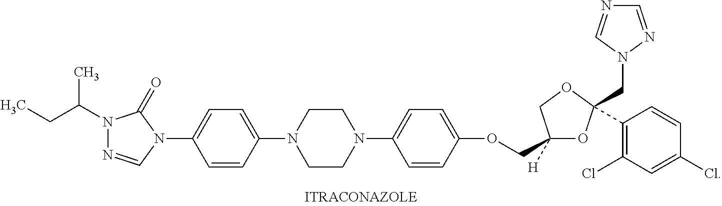

Terconazole, available from Janssen Pharmaceutica, N.V., Beerse, Belgium and described in U.S. Pat. No. 4,223,036, is practically insoluble (<0.1 mg/ml) in water has the following structure:

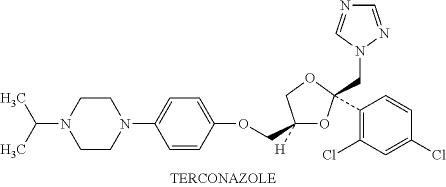

Fluconazole, available from Pfizer, New York, N.Y. and described in U.S. Pat. No. 6,790,957, is practically insoluble in water (about 1 ug/ml) and has the following structure:

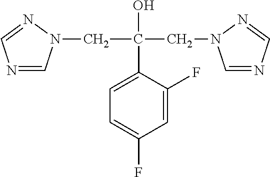

Voriconazole (formerly UK 109496), available from Pfizer, New York, N.Y. and described in U.S. Pat. No. 5,278,175, has the following structure:

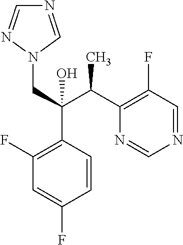

Letrozole, described in U.S. Pat. No. 4,978,672, is practically insoluble in water and has the following structure:

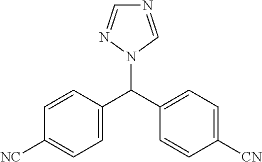

Ravuconazole (BMS-207147; formerly ER-30346), available from Bristol-Myers Squibb, Princeton, N.J. and described in U.S. Pat. No. 5,648,372, has the following structure:

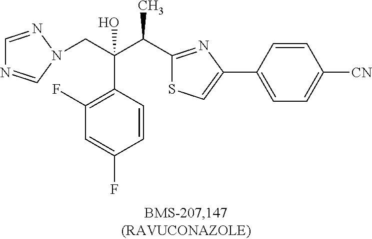

Ketoconazole, described in U.S. Pat. No. 4,144,346, has the following structure:

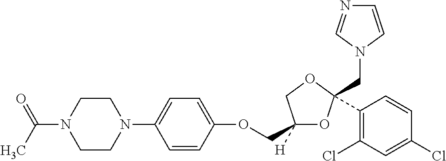

Epirazole (Omeprazole), described in U.S. Pat. No. 4,255,431, has the following structure:

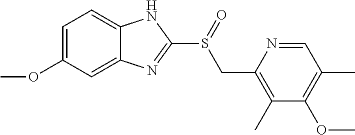

Saperconazole, available from Janssen Pharmaceutica, N.V., Beerse, Belgium and described in U.S. Pat. No. 4,916,134, which is poorly soluble in water has the following structure:

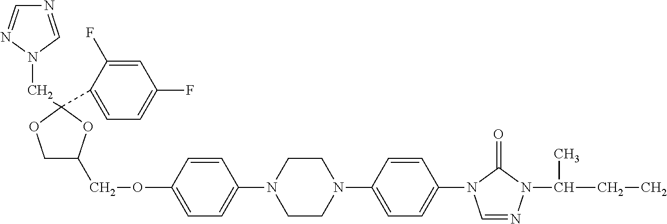

Difenamizole, described in JP 68 6621, which is practically insoluble in water has the following structure:

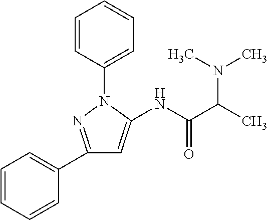

Compositions of the invention are room temperature solids comprising an azole antifungal compound molecularly dispersed in a pH sensitive polymer. Suitable polymers for use in preparing compositions of the inventin include, but are not limited to, cellulose acetate phthalate, polyvinyl acetate phthalate, alginic acid, a methacrylic acid polymer, a carbomer, polacrillin, chitosan, or a hydroxypropylmethylcellulose-derivatve polymer, for example, hypromellose-phthalate (hydroxypropylmethylcellulose-phthalate) or hypromellose acetate succinate (hydroxypropylmethylcellulose acetate succinate, also designated herein for convenience, HPMCAS), prefererably the polymer is HPMCAS Hypromellose acetate succinate (hydroxypropyl methylcellulose acetate succinate; HPMCAS) is commercially available, for example, the AQOAT series of polymers available from Shin-Etsu, which provides HPMCAS polymer in different grades, for example, L, M, and H grades. HPMCAS polymers have solubility varying from pH≧5.5 to 6.8. Specifically, grade L is soluble at pH≧5.5, grade M is soluble at pH≧6, and grade H is soluble at pH≧6.8. Both granular (type G) and micronized (type F) forms of AQOAT HPMCAS are available as and either are suitable for use in the present invention. In some embodiments it is preferred to prepare a composition of the invention using AQOAT HPMCAS grade L wherein the acetyl content is stated to be in the range of 7% to 11% and the range of succinoyl content is stated to be in the range of 10% to 14%. In some embodiments it is preferred to prepare a composition of the invention using AQOAT HPMCAS grade M wherein the acetyl content is stated to be in the range of 5% to 9% and the range of succinoyl content is stated to be in the range of 14% to 18%. As used herein, the terms “grade L” and “grade M” refer to grades of HPMCAS that are consistent with the AQOAT line of HPMCAS grade L and HPMCAS grade M available from Shin-Etsu.

In especially preferred embodiments, posaconazole is used as the azole antifungal compound. In certain embodiments, posaconazole in an amorphous form is stable within the molecular solid dispersions disclosed herein after storage at 15-25° C. and 50-60% relative humidity for at least one year. In certain other embodiments, posaconazole in an amorphous form is stable within the molecular solid dispersions disclosed herein after storage at 40° C. and 75% relative humidity for at least 3 months.

In some embodiments it is preferred to prepare a composition of the invention using HPMCAS and posaconazole, wherein the weight ratio of HPMCAS:posaconazole present in the molecular solid dispersion provided is from about 1:1 w/w HPMCAS/posaconazole to about 10:1 w/w HPMCAS/posaconazole, preferably the weight ratio of HPMCAS:posaconazole in the molecular solid dispersion provided is from about 2:1 w/w HPMCAS/posaconazole to about 4:1 w/w HPMCAS/posaconazole, more preferably the weight ratio of HPMCAS:posaconazole in the molecular solid dispersion provided is about 3:1 w/w HPMCAS/posaconazole. In some embodiments it is preferred to provide a molecular solid dispersions of the invention comprising HPMCAS polymer and posaconazole wherein weight percentage of HPMCAS polymer:posaconazole is from about 40 wt. % HPMCAS/60 wt. % posaconazole to about 95 wt. % HPMCAS/5 wt. % posaconazole.

In some embodiments it is preferred to incorporate a molecular solid dispersions of the present invention into a pharmaceutical composition suitable for oral administration, herein termed “oral” pharmaceutical compositions. Suitable dosage forms comprising oral pharmaceutical compositions include, but are not limited to, capsules, tablets, granules, powders, and unit dose packets. In some embodiments oral pharmaceutical compositions may optionally additionally comprise one or more pharmaceutically acceptable excipients. In certain embodiments, oral dosage forms as described herein have a drug loading capacity of at least 50 mg, 75 mg, 100 mg, or 125 mg per oral dosage form.

Suitable pharmaceutically acceptable excipients are well known in the art. Generally, excipients include, but are not limited to, surface active agents (e.g., sodium lauryl sulfate and polysorbate 80), drug complexing agents or solubilizers (e.g., polyethylene glycols, caffeine, xanthene, gentisic acid and cylodextrins), diluents (e.g., lactose, mannitol, xylitol, microcrystalline cellulose, calcium diphosphate, and starch), disintegrants (e.g., sodium starch glycolate, sodium alginate, carboxymethyl cellulose sodium, methyl cellulose, low-substituted hydroxypropylcellulose (L-HPC (such as LH-21, and LH-B1) and croscarmellose sodium), glidants (e.g., silicon dioxide and Talc), binders (e.g., methyl cellulose, microcrystalline cellulose, starch, and gums such as guar gum, and tragacanth), lubricants(e.g., magnesium stearate and calcium stearate), pH modifiers (e.g., citric acid, acetic acid, ascorbic acid, lactic acid, aspartic acid, succinic acid, and phosphoric acid), anti-oxidants (e.g., ascorbic acid, butylated hydroxytoluene, and butylated hydroxyanisole), pigments, and flavorants which may be used for customary purposes and in typical amounts without affecting the properties of the compositions. These excipients may be mixed or granulated with weakly basic and poorly-aqueous soluble azole and HPMCAS, or may be added after weakly basic and poorly-aqueous soluble azole and HPMCAS are mixed or granulated, in order to formulate the composition into a suitable oral composition.

### Methods of Preparing Molecular Solid Dispersions

Another aspect of the invention provides methods of preparing the molecular solid dispersions according to the present invention. In certain embodiments, molecular solid dispersions comprising a weakly basic and poorly-aqueous soluble azole, preferably posaconazole, and a pH sensitive polymer, preferably HPMCAS polymer, may be prepared by dissolving both the weakly basic and poorly-aqueous soluble azole and the polymer in an organic solvent followed by evaporation of the organic solvent. In some embodiments employing posaconazole as the azole and HPMCAS as the pH sensitive polymer, the dissolution step may be carried out at a temperature of from about 25° C. to about 70° C. Subsequent evaporation of the organic solvent may be accomplished either at elevated temperature or by spray-drying. When the evaporation step is carried out by spray-drying, suitable temperatures may be in the range of about 30° C. to about 80° C. In some embodiments it is preferred to employ as an organic solvent, methanol, ethanol, isopropanol, or acetone, or a combination of two or more thereof. In some embodiments it is preferred to use as an organic solvent a combination of ethanol and acetone or methanol and acetone.

In some especially preferred embodiments it is preferred to provide a molecular solid dispersion of the present invention comprising posaconazole and HPMCAS by dissolving both posaconazole and HPMCAS in an organic solvent followed by evaporation of the organic solvent. In one embodiment, the ratio by weight of HPMCAS to posaconazole is in the range of about 1:1 to about 10:1. In one embodiment, the ratio by weight of HPMCAS to posaconazole is in the range of about 2:1 to about 4:1. In a certain preferred embodiment, the ratio by weight of HPMCAS to posaconazole is about 3:1. In another embodiment, the molecular solid dispersions are in the range (% w/w) of 40-95% HPMCAS to 5-60% posaconazole. Dissolution of posaconazole and HPMCAS in the organic solvent may be accomplished at a temperature in the range of 25-70° C. Subsequent evaporation of the organic solvent may be accomplished either at elevated temperature or by spray drying. Suitable temperatures may be in the range of 30-80° C. Suitable organic solvents include, but are not limited to, methanol, ethanol, isopropanol, acetone, or a combination of two or more thereof. In certain embodiments, a combination of organic solvents may be used, such as ethanol and acetone or methanol and acetone. Such combinations may be in any appropriate ratio in the range of 1:99 to 99:1 volume to volume. In certain alternative embodiments, molecular solid dispersions comprising posaconazole and HPMCAS as disclosed herein may be prepared using hot melt extrusion.

Methods of Treatment and/or Prevention of Fungal Infections

Another aspect of the invention provides methods for the treatment and/or prevention of a condition, for example, Otomycosis or Chromomycosis, or a fungal infection in a patient comprising administering orally to the patient in need thereof a pharmaceutical composition comprising a solid molecular dispersion of an azole antifungal compound in a polymer. In some embodiments it is preferred for the solid molecular dispersion of the invention to comprise posaconazole. Fungal infections which may be treated with a pharmaceutical formulation comprising a solid molecular dispersion of the invention include, but are not limited to, Asperillus sp. and Candida sp. (including Candida albicans), as well as those caused by zygomycetes (including Mucor, Rhizopus, Rhizomucor etc) and/or dermatophytes (including Tinea sp., (e.g., Tinea corporis, Tinea cruris, Tinea pedis, Tinea barbae, Tinea capitis, Tinea nigra, Tinea favosa, Tinea Imbricata), Trichophyton sp., (e.g., Trichophyton rubrum, Trichophyton interdigitale, Trichophyton tonsurans, Trichophyton soudanense, and Trichophyton violaceum), Epidermophyton sp. (e.g., Epidermophyton floccosum), and Microsporum sp. (e.g., Microsporum gypseum)) or any other agent associated with onychomycosis. Notably, NOXAFIL™ (posaconazole) is currently indicated for prophylaxis of invasive Aspergillus and Candida infections in patients, 13 years of age and older, who are at high risk of developing these infections due to being severely immunocomprised, such as hematopoietic stem cell transplant (HSCT) recipients with graft-versus-host disease (GVHD) or those with hematologic malignancies with prolonged neutropenia from chemotherapy. NOXAFIL™ (posaconazole) is also indicated for the treatment of oropharyngeal candidiasis, including oropharyngeal candidiasis refractory to itraconazole and/or fluconazole.

In certain embodiments, the pharmaceutical compositions described herein may be administered to a patient in need thereof every 8 hours, every 12 hours, or every 24 hours. In certain embodiments a dose is administered to a patient in need thereof every 12 hours, or every 24 hours.

In some embodiments it is preferred for a solid molecular dispersions comprising posaconazole and HPMCAS described herein to be administered to a patient in need thereof thrice per day (TID), twice a day (BID), or once a day (QD); more preferably, twice a day (BID), or once a day (QD). In one embodiment, a pharmaceutical composition of the invention comprising posaconazole is administered to a patient in need thereof every 8 hours, 12 hours, or every 24 hours.

In certain such embodiments, a dose comprises at least one oral dosage form. In certain such embodiments, a dose may comprise at least one additional oral dosage form administered simultaneously with the first dosage form, or within about 5 minutes, or even ten minutes of the first oral dosage form.

The pharmaceutical compositions of the present invention can be administered to a patient according to a dosing regimen. It should be understood that the specific dosing regimen for any particular patient will depend on a variety of factors, including species, age, body weight, body surface area, height, general health, sex, diet, time of administration, rate of excretion, drug combination, specific disease being treated, the severity of the condition, the renal and hepatic function of the patient, the particular active ingredient employed, and the judgment of the treating physician.

Other features and embodiments of the invention will become apparent by the following examples which are given for illustration of the invention rather than limiting its intended scope.

### Examples

### Preparation of Molecular Solid Dispersions

Two exemplary molecular solid dispersions of posaconazole with HPMCAS of the present invention are detailed in Table 1. The molecular solid dispersions referred to therein as Capsule A and Capsule B were made by dissolving both posaconazole and micronized HPMCAS of grade L or grade M, respectively, in a mixed solvent of ethanol/acetone or methanol/acetone in pre-determined ratios as detailed in Table 1 at a temperature range of between 25-70° C. with vigorous agitation. The solvents were then evaporated at 30-80° C. to form a molecular solid dispersion material. Alternatively, the solvents may be evaporated by spray drying.

TABLE 1
Exemplary molecular solid dispersions of
posaconazole with HPMCAS

| Ingredient | Capsule A | Capsule B |
|---|---|---|
| Posaconazole | 25 mg | 25 mg |
| HPMCAS grade L | 75 mg | |
| HPMCAS grade M | | 75 mg |

Dissolution Profile of Molecular Solid Dispersions of Posaconazole with HPMCAS

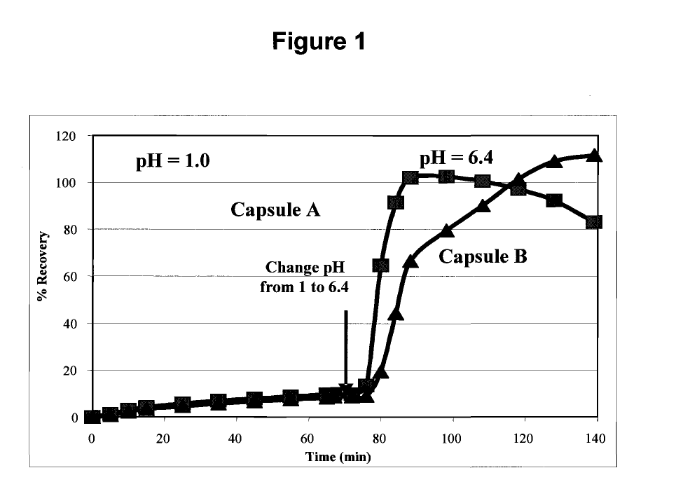

The dissolution profile of molecular solid dispersions of posaconazole in Capsule A and Capsule B reflected in FIG. 1 was determined as follows. In brief, a USP-II Apparatus Paddle Stirrer was used for dissolution testing. A sample dissolution profile was obtained in 900 ml of dissolution medium at pH 1 comprising 0.1 N HCl using the USP-II Apparatus Paddle Stirrer, without sinkers, operated at 100 RPM. The dissolution medium was subsequently adjusted to 6.4 by the addition of 100 ml of sodium phosphate to make up a 50 mM sodium phosphate buffer. Aliquots were assayed on-line (i.e., aliquots were returned for subsequent assaying) using a UV-spectrometer at a wavelength of 245 nm for posaconazole at several time points both at pH 1 as well as following the change of pH to 6.4 as reflected in FIG. 1.

The dissolution profiles of two molecular solid dispersions of posaconazole with HPCMAS (grade L or grade M) according to the present invention described herein as Capsule A and Capsule B respectively are illustrated in FIG. 1. As illustrated in FIG. 1, “% recovery” refers to the amount of posaconazole released from the samples relative to the total amount of posaconazole in the samples being assayed. Controlled release of posaconazole from both molecular solid dispersions was observed at pH 1. Even though at pH 1, posaconazole has a solubility of 790 μg/mL in aqueous solution, posaconazole was minimally released from molecular solid dispersions with HPMCAS when the dissolution media was at pH 1.

Surprisingly, molecular solid dispersions with HPMCAS also prevented posaconazole precipitation at pH>4 (where posaconazole has a solubility of less than 1 μg/mL in aqueous solution). Moreover, the dissolution profile of the molecular solid dispersions at pH 6.4 was sustained for a prolonged period of time (see FIG. 1). Consequently, the present inventors believe that surprisingly the molecular solid dispersions of their invention will be beneficial to increase both the rate and/or extent of posaconazole absorption in the gastrointestinal tract thereby leading to enhanced bioavailability of posaconazole.

### X-Ray Power Diffraction of Molecular Solid Dispersions

The presence of crystalline forms of posaconazole was assessed using X-ray powder diffraction as described in U.S. Pat. No. 6,713,481. Molecular solid dispersions of posaconazole with HPMCAS at ratios ranging from 1:1 to 3:1 (by weight) were assessed for the presence of crystalline posaconazole after storage under RH4 conditions (i.e., 40° C. and 75% relative humidity) for 3 months or after storage under ambient conditions (i.e., 15-25° C. and 50-60% relative humidity) for 1 year. Specifically, molecular solid dispersions of HPMCAS grade M:posaconazole at ratios of 1:1, 2:1, and 3:1 (by weight) were examined. Similarly, molecular solid dispersions of HPMCAS grade L:posaconazole at ratios of 2:1 and 3:1 were also examined. No crystalline posaconazole was detected in any of these molecular solid dispersions at either timepoint. Notably, the detection limit for crystalline posaconazole using this assay is less than 3%. These results indicate that posaconazole in an amorphous form is stable within these molecular solid dispersions.

### Bioavailability Comparison of Posaconazole Compositions

Adult male monkeys (6 monkeys/group) were dosed after an overnight fast with one of three different oral compositions as illustrated in Table 2. Food was returned at 4 hrs post-dose and body weight was recorded on the day of dosing. Blood was collected from each monkey pre-dose, as well as 0.25, 0.5, 1, 2, 4, 8, 24, 48, 72, 96, and 120 hr post-dose for determining the concentration of posaconazole in the plasma and to calculate the pharmacokinetic (PK) parameters for each composition.

The three different posaconazole compositions examined were an oral suspension (described in U.S. Publication No. 2003/0055067) as well as capsule dosage forms (Capsule A and Capsule B) of molecular solid dispersions.

TABLE 2
Dosing of Posaconazole Compositions in Monkeys

| | | | Actual Mean | Actual Mean |
|---|---|---|---|---|
| | | Dosing | Dose | Dose |
| Group | | Composition | Administered | Administered |
| No. | Composition | Concentration | (mg) | (mg/kg) (*CV) |
| 1 | Oral | 40 mg/mL | 60 | 13.2 (4) |
| | Suspension | | | |
| 2 | Capsule A | 60 mg/capsule | 60 | 16.1 (9) |
| 3 | Capsule B | 60 mg/capsule | 60 | 16.1 (10) |

*CV = the coefficient of variation defined as the ratio of the standard deviation to the mean

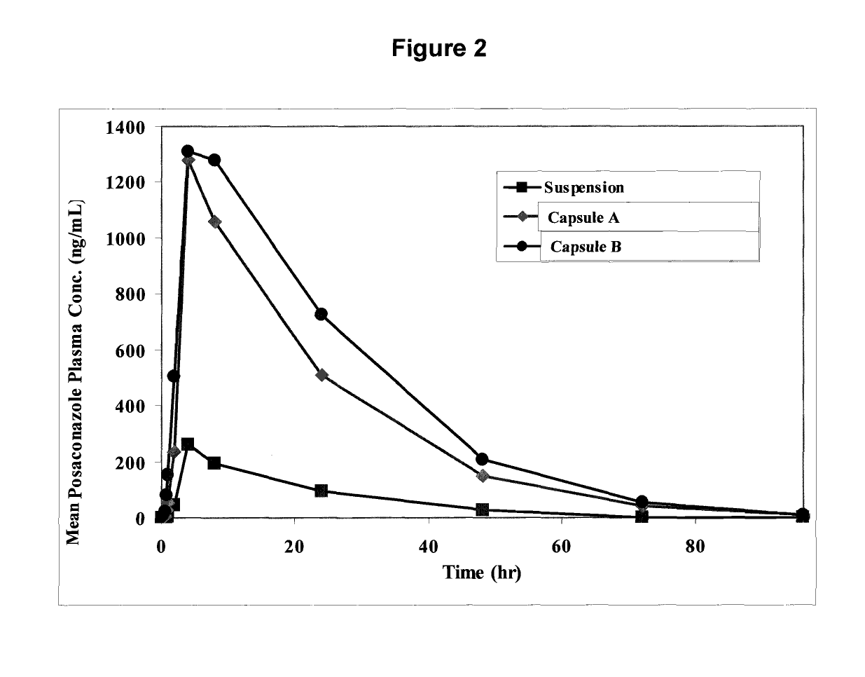

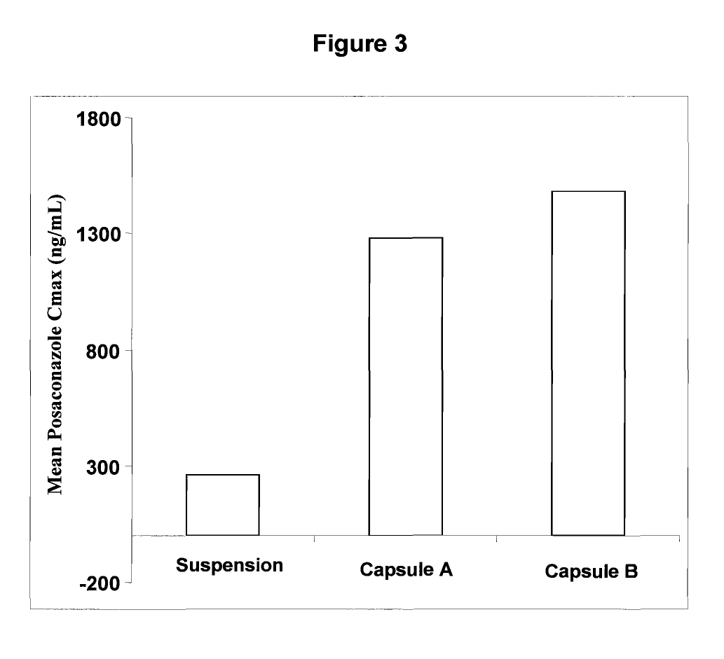

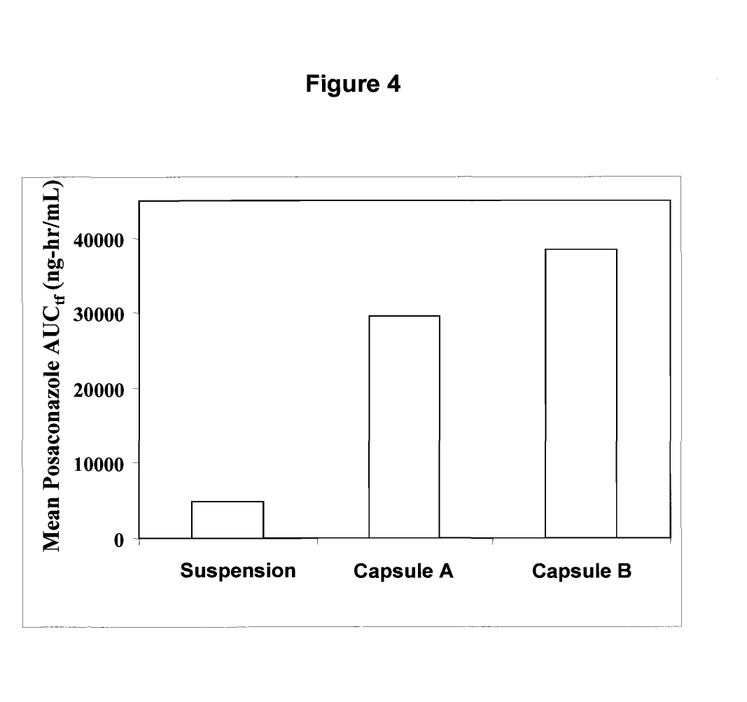

The resulting mean plasma concentration for each composition over time is displayed graphically in FIG. 2. In addition, the mean Cmax and exposure (AUC(tf)) for each composition is shown in FIGS. 3 and 4, respectively. Likewise, the mean (CV, %) AUC (tf), AUC/dose, and CL/F for each posaconazole composition examined is summarized in Table 3 below.

TABLE 3
Mean (CV, %) pharmacokinetic parameters for
posaconazole compositions.
Mean (*CV, %) Pharmacokinetic Parameters
Posaconazole

| | Oral Administration | | | | | |
|---|---|---|---|---|---|---|
| | Oral | | | | | |
| Parameter | Suspension | | Capsule A | | Capsule B | |
| AUC(tf)^{a} | 4950 | (30) | 29600 | (24) | 38400 | (28) |
| AUC(tf)/Dose^{b} | 373 | (29) | 1860 | (27) | 2390 | (26) |
| CL/F^{c} | 11.8 | (29) | 2.11 | (24) | 1.66 | (30) |

*CV = the coefficient of variation defined as the ratio of the standard deviation to the mean
^{a}AUC(tf) = ng-hr/mL
^{b}Dose adjusted, Normalized to 1 mg/Kg dose; expressed as (ng-hr/mL)/(mg/kg)
^{c}CL/F = L/hr

In addition, the individual and mean (CV, %) plasma concentrations and pharmacokinetic parameters for each posaconazole composition examined is detailed in Tables 4-6 below.

| | | | | | | | | |
|---|---|---|---|---|---|---|---|---|
| Tmax (hr) | 4 | 4 | 4 | 4 | 4 | 4 | 4.00 | (4-4) |
| AUC (tf) | 6890 | 4550 | 3140 | 3990 | 6650 | 4480 | 4950 | (30) |
| (ng-hr/mL) | | | | | | | | |
| tf (hr) | 48 | 48 | 48 | 48 | 48 | 48 | 48.0 | (0) |
| t½ (hr) | 16.7 | 9.88 | 14.6 | 15.4 | 11.4 | 14.6 | 13.7 | (19) |
| CL/F (L/hr) | 7.61 | 12.7 | 17.0 | 13.2 | 8.48 | 11.9 | 11.8 | (29) |
| Dose (mg/kg) | 12.9 | 13.0 | 13.2 | 12.8 | 14.2 | 13.2 | 13.2 | (4) |
| AUC/Dose^{b} | 534 | 350 | 238 | 312 | 468 | 339 | 373 | (29) |

*CV = the coefficient of variation defined as the ratio of the standard deviation to the mean
NC = Not calculated
^{a}Tmax expressed as median (min-max)
^{b}Normalized to 1 mg dose (mg/kg); expressed as (ng-hr/mL)/(mg/kg)

TABLE 5
Individual and mean (CV, %) plasma concentration and pharmacokinetic
parameters for posaconazole following a single oral administration of 60 mg posaconazole to
male cynomolgus monkeys in Capsule A (molecular solid dispersion with HPMCAS grade L).

| | ng Posaconazole/mL | | | | | | | |
|---|---|---|---|---|---|---|---|---|
| | | | | | | | Mean³ | |
| Time Point | Cynomolgus Monkey No. | | | | | | (n = 6) | |
| (hr) | 2001 | 2002 | 2003 | 2004 | 2005 | 2006 | *CV (%) | |
| Pre-dose | 0 | 0 | 0 | 0 | 0 | 0 | 0 | (NC) |
| 0.25 | 0 | 0 | 0 | 15.6 | 0 | 0 | 2.60 | (245) |
| 0.5 | 0 | 0 | 0 | 32.1 | 0 | 0 | 5.35 | (245) |
| 0.75 | 0 | 11.6 | 0 | 130 | 24.2 | 0 | 27.6 | (185) |
| 1 | 0 | 33.5 | 0 | 213 | 44.3 | 28.4 | 53.2 | (151) |
| 2 | 61.1 | 234 | 62.6 | 551 | 219 | 269 | 233 | (77) |
| 4 | 979 | 1400 | 1290 | 1460 | 1410 | 1130 | 1280 | (15) |
| 8 | 904 | 1350 | 1010 | 1290 | 995 | 831 | 1060 | (20) |
| 24 | 423 | 662 | 346 | 544 | 630 | 445 | 508 | (24) |
| 48 | 100 | 171 | 56.5 | 257 | 210 | 104 | 150 | (51) |
| 72 | 26.3 | 51.9 | 10.5 | 72.2 | 63.8 | 17.8 | 40.4 | (64) |
| 96 | 0 | 13.9 | 0 | 16.7 | 15.3 | 0 | 7.65 | (110) |
| 120 | 0 | 0 | 0 | 0 | 0 | 0 | 0 | (NC) |
| Cmax | 979 | 1400 | 1290 | 1460 | 1410 | 1130 | 1280 | (15) |
| (ng/mL) | | | | | | | | |
| Tmax (hr) | 4 | 4 | 4 | 4 | 4 | 4 | 4.00 | (4-4) |
| AUC (tf) | 23200 | 36800 | 22500 | 37300 | 33900 | 23700 | 29600 | (24) |
| tf (hr) | 72 | 96 | 72 | 96 | 96 | 72 | 84.0 | (16) |
| t½ (hr) | 12.0 | 13.0 | 9.52 | 12.2 | 12.7 | 10.3 | 11.6 | (12) |
| CL/F (L/hr) | 2.53 | 1.62 | 2.65 | 1.60 | 1.76 | 2.50 | 2.11 | (24) |
| Dose | 15.3 | 16.9 | 15.2 | 14.4 | 16.1 | 18.5 | 16.1 | (9) |
| (mg/kg) | | | | | | | | |
| AUC/Dose^{b} | 1520 | 2180 | 1480 | 2590 | 2110 | 1280 | 1860 | (27) |

*CV = the coefficient of variation defined as the ratio of the standard deviation to the mean
NC = Not calculated
^{a}Tmax expressed as median (min-max)
^{b}Normalized to 1 mg dose (mg/kg); expressed as (ng-hr/mL)/(mg/kg)

TABLE 6
Individual and mean (CV, %) plasma concentration and pharmacokinetic
parameters for posaconazole following a single oral administration of 60 mg posaconazole to
male cynomolgus monkeys in Capsule B (molecular solid dispersion with HPMCAS grade M).

| | ng Posaconazole/mL | | | | | | | |
|---|---|---|---|---|---|---|---|---|
| | | | | | | | Mean³ | |
| Time Point | Cynomolgus Monkey No. | | | | | | (n = 6) | |
| (hr) | 3001 | 3002 | 3003 | 3004 | 3005 | 3006 | *CV (%) | |
| Pre-dose | 0 | 0 | 0 | 0 | 0 | 0 | 0 | (NC) |
| 0.25 | 0 | 0 | 0 | 23.6 | 0 | 0 | 3.93 | (245) |
| 0.5 | 0 | 64.7 | 0 | 30.7 | 0 | 42.5 | 23.0 | (119) |
| 0.75 | 0 | 287 | 0 | 37.8 | 0 | 167 | 82.0 | (146) |
| 1 | 0 | 522 | 0 | 97.6 | 0 | 312 | 155 | (140) |
| 2 | 174 | 1080 | 101 | 654 | 195 | 805 | 502 | (80) |
| 4 | 608 | 1690 | 1090 | 1580 | 1020 | 1840 | 1310 | (36) |
| 8 | 1610 | 1400 | 1120 | 1570 | 915 | 1080 | 1280 | (22) |
| 24 | 1010 | 877 | 581 | 854 | 444 | 594 | 727 | (30) |
| 48 | 396 | 234 | 134 | 261 | 103 | 123 | 209 | (54) |
| 72 | 123 | 63.0 | 24.6 | 61.6 | 23.3 | 25.8 | 53.6 | (72) |
| 96 | 27.7 | 14.7 | 0 | 17.2 | 0 | 0 | 9.93 | (118) |
| 120 | 0 | 0 | 0 | 0 | 0 | 0 | 0 | (NC) |
| Cmax | 1610 | 1690 | 1120 | 1580 | 1020 | 1840 | 1480 | (22) |
| (ng/mL) | | | | | | | | |
| Tmax (hr) | 8 | 4 | 8 | 4 | 4 | 4 | 4.00 | (4-8) |
| AUC (tf) | 51200 | 45900 | 29800 | 46500 | 24100 | 32900 | 38400 | (28) |
| (ng-hr/mL) | | | | | | | | |
| tf (hr) | 96 | 96 | 72 | 96 | 72 | 72 | 84.0 | (16) |
| t½ (hr) | 12.5 | 12.0 | 10.5 | 12.2 | 11.3 | 10.6 | 11.5 | (7) |
| CL/F (L/hr) | 1.16 | 1.30 | 1.99 | 1.28 | 2.45 | 1.80 | 1.66 | (30) |
| Dose | 17.9 | 16.1 | 14.1 | 15.7 | 18.1 | 14.7 | 16.1 | (10) |
| (mg/kg) | | | | | | | | |
| AUC/Dose^{b} | 2860 | 2850 | 2110 | 2960 | 1330 | 2240 | 2390 | (26) |

*CV = the coefficient of variation defined as the ratio of the standard deviation to the mean
NC = Not calculated
^{a}Tmax expressed as median (min-max)
^{b}Normalized to 1 mg dose (mg/kg); expressed as (ng-hr/mL)/(mg/kg)

Notably, molecular solid dispersions with HPMCAS increased posaconazole exposure when compared to the oral suspension indicating an increase in enhanced bioavailability. In fact, the difference in AUC(tf) was about 6 times and about 8 times for Capsule A (with HPMCAS grade L) and Capsule B (with HPMCAS grade M), respectively, compared to the oral suspension.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

1. A composition comprising a weakly basic and poorly-aqueous soluble azole molecularly dispersed in at least one pH sensitive polymer prepared by evaporating the solvent from a solution comprising the azole and said at least one pH sensitive polymer.

2. A composition comprising posaconazole molecularly dispersed in hypromellose acetate succinate prepared by evaporating the solvent from a solution comprising posaconazole and the hypermellose acetate succinate polymer.

3. A pharmaceutical composition suitable for oral administration comprising the composition of claim 2.

4. A method for the treatment or prevention of a fungal infection, the method comprising administering to a patient in need thereof an oral pharmaceutical composition comprising a solid molecular dispersion prepared by a spray-drying technique, wherein the solid molecular dispersion comprises:
(a) posaconazole; and
(b) hypromellose acetate succinate (HPMCAS).

5. The method of claim 4, wherein the fungal infection is an Aspergillus or a Candida infection, or is a fungal infection caused by a zygomycetes or a dermatophyte.
